(19) United States
(12) Patent Application Publication
(10) Pub. No.: US 2023/0200836 A1
(43) Pub. Date: Jun. 29, 2023

(54) ULTRASONIC TREATMENT TOOL

(71) Applicant: Olympus Corporation, Tokyo (JP)

(72) Inventors: Akinori KOBAYASHI, Tokyo (JP); Satoshi MIYAJIRI, Tokyo (JP); Akihito KANO, Tokyo (JP); Tsubasa NIIYAMA, Tokyo (JP); Yusuke INUI, Tokyo (JP)

(21) Appl. No.: 18/176,030

(22) Filed: Feb. 28, 2023

Related U.S. Application Data
(63) Continuation of application No. PCT/JP2020/034600, filed on Sep. 11, 2020.

(51) Int. Cl. A61B 17/32

(52) U.S. Cl. CPC A61B 17/320068; A61B 2017/320089

(57) ABSTRACT

An ultrasonic treatment tool includes: an ultrasonic oscillator in which a plurality of piezoelectric elements configured to generate ultrasonic oscillation are arranged along a longitudinal axis direction of the ultrasonic oscillator, the ultrasonic oscillator being configured to transmit the ultrasonic oscillation from a proximal end side to a distal end side of the ultrasonic oscillator; a contact receiver configured to transmit power and a control signal to the ultrasonic oscillator; and a floating structure configured to suppress transmission of ultrasonic oscillation generated by the ultrasonic oscillator to the contact receiver or dampen the ultrasonic oscillation.

### CROSS-REFERENCE TO RELATED APPLICATION

This application is a continuation of International Application No. PCT/JP2020/034600, filed on Sep. 11, 2020, the entire contents of which are incorporated herein by reference.

### BACKGROUND

### 1. Technical Field

The present disclosure relates to an ultrasonic treatment tool.

### 2. Related Art

In the related art, there has been known an ultrasonic treatment tool used for the treatment of a treatment target site in a biological tissue by applying energy such as ultrasonic waves to the target site (refer to JP 6257886 B2, for example).

The ultrasonic treatment tool described in JP 6257886 B2 includes an ultrasonic oscillator, a central housing that accommodates the ultrasonic oscillator, a nose cone that holds the ultrasonic oscillator in cooperation with the central housing, and a cover that is an exterior member, and also includes a keyed ring for the purpose of sound insulation or the like, as well as an O-ring and the

### SUMMARY

In some embodiments, an ultrasonic treatment tool includes: an ultrasonic oscillator in which a plurality of piezoelectric elements configured to generate ultrasonic oscillation are arranged along a longitudinal axis direction of the ultrasonic oscillator, the ultrasonic oscillator including a flange at a node position of ultrasonic oscillation in a resonance state, the ultrasonic oscillator being configured to transmit the ultrasonic oscillation from a proximal end side to a distal end side of the ultrasonic oscillator; a contact receiver configured to transmit power and a control signal to the ultrasonic oscillator; and a floating structure configured to suppress transmission of ultrasonic oscillation generated by the ultrasonic oscillator to the contact receiver or dampen the ultrasonic oscillation, the floating structure including: an oscillation damping material configured to dampen the ultrasonic oscillation transmitted from the flange to the contact receiver; a storage configured to accommodate the ultrasonic oscillator; and a holder that is inserted into the contact receiver and the storage, the holder being configured to hold the flange of the ultrasonic oscillator in cooperation with the storage, the oscillation damping material being formed of a material that is softer than the holder and that is filled in a gap at a portion where the contact receiver and the holder are in contact with each other.

In some embodiments, an ultrasonic treatment tool includes: an ultrasonic oscillator in which a plurality of piezoelectric elements configured to generate ultrasonic oscillation are arranged along a longitudinal axis direction of the ultrasonic oscillator, the ultrasonic oscillator being configured to transmit the ultrasonic oscillation from a proximal end side to a distal end side of the ultrasonic oscillator; a contact receiver configured to transmit power and a control signal to the ultrasonic oscillator; and a floating structure configured to suppress transmission of ultrasonic oscillation generated by the ultrasonic oscillator to the contact receiver or dampen the ultrasonic oscillation, the floating structure including a cover that includes an oscillation damping material, the cover being configured to hold the proximal end side of the ultrasonic oscillator, the cover being configured to hold an earphone jack functioning as the contact receiver on a side facing a side holding the ultrasonic oscillator, the cover including: a main body formed of a rubber material and covering the proximal end side of the ultrasonic oscillator; and a flexible substrate covering an outer circumference of the main body, the flexible substrate being configured to transmit the power and the control signal to the ultrasonic oscillator.

In some embodiments, an ultrasonic treatment tool includes: an ultrasonic oscillator in which a plurality of piezoelectric elements configured to generate ultrasonic oscillation are arranged along a longitudinal axis direction of the ultrasonic oscillator, the ultrasonic oscillator being configured to transmit the ultrasonic oscillation from a proximal end side to a distal end side of the ultrasonic oscillator; a contact receiver configured to transmit power and a control signal to the ultrasonic oscillator; and a floating structure configured to suppress transmission of ultrasonic oscillation generated by the ultrasonic oscillator to the contact receiver or dampen the ultrasonic oscillation, the floating structure including a cover that includes an oscillation damping material, the cover being configured to hold the proximal end side of the ultrasonic oscillator, the cover being configured to hold an earphone jack functioning as the contact receiver on a side facing a side holding the ultrasonic oscillator, the cover including: a main body formed of a resin and covering the proximal end side of the ultrasonic oscillator; a flexible substrate covering an outer circumference of the main body, the flexible substrate being configured to transmit the power and the control signal to the ultrasonic oscillator; and the oscillation damping material disposed between the ultrasonic oscillator and the main body.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a block diagram schematically illustrating an overall configuration of an ultrasonic treatment system according to a first embodiment of the disclosure;

FIG. 2 is a cross-sectional view of an ultrasonic transducer used in the ultrasonic treatment system of FIG. 1;

FIG. 3 is a diagram illustrating connection between an ultrasonic transducer and a contact unit;

FIG. 4 is a cross-sectional view of an ultrasonic transducer according to a second embodiment of the disclosure;

FIG. 5 is a cross-sectional view of an ultrasonic transducer according to a modification of the second embodiment of the disclosure;

FIG. 6 is a cross-sectional view of an ultrasonic transducer according to a third embodiment of the disclosure;

FIG. 7 is a cross-sectional view of an ultrasonic transducer according to a modification of the third embodiment of the disclosure;

FIG. 8A is a side view of an ultrasonic transducer according to a fourth embodiment of the disclosure;

FIG. 8B is a cross-sectional view of the ultrasonic transducer according to the fourth embodiment of the disclosure;

FIG. 9 is an exploded perspective view of an ultrasonic transducer according to a fifth embodiment of the disclosure;

FIG. 10 is a cross-sectional view of the ultrasonic transducer according to the fifth embodiment of the disclosure;

FIG. 11 is a partial cross-sectional view of an ultrasonic treatment tool according to a sixth embodiment of the disclosure;

FIG. 12 is a cross-sectional view of an ultrasonic oscillator and a cover used in the ultrasonic treatment tool of FIG. 11;

FIG. 13 is a cross-sectional view of an ultrasonic oscillator and a cover according to a first modification of the sixth embodiment of the disclosure;

FIG. 14 is a cross-sectional view of an ultrasonic oscillator and a cover according to a second modification of the sixth embodiment of the disclosure;

FIG. 15 is a cross-sectional view of an ultrasonic oscillator and a cover according to a third modification of the sixth embodiment of the disclosure; and

FIG. 16 is a side view 16 of an ultrasonic transducer according to a seventh embodiment of the disclosure.

### DETAILED DESCRIPTION

The following will describe an ultrasonic treatment system including an ultrasonic transducer as a mode for implementing the disclosure (hereinafter, referred to as an “embodiment”). The disclosure is not limited by the present embodiment. In the drawings, same reference signs are attached to the same portions. Furthermore, it needs to be kept in mind that the drawings are schematic, and the relationship between the thickness and the width of individual members and the ratio between the members are different from an actual case. Still further, there are portions having different ratios even between the drawings.

### First Embodiment

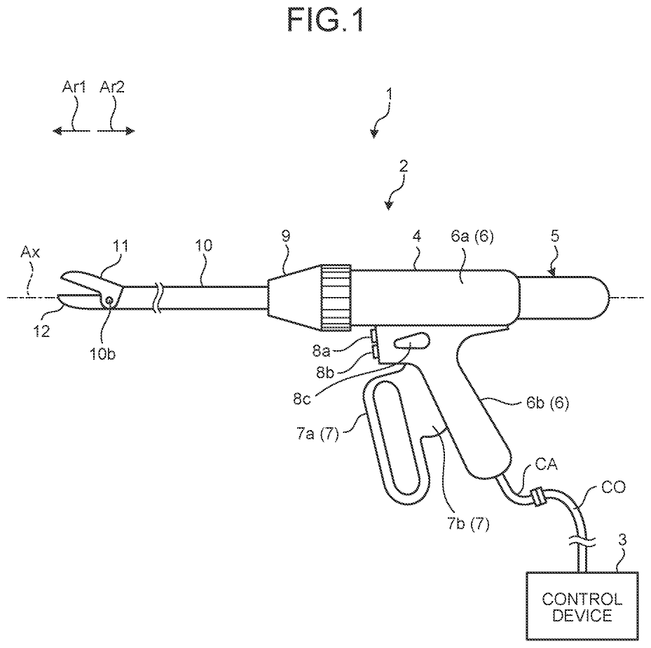

FIG. 1 is a block diagram schematically illustrating an overall configuration of an ultrasonic treatment system according to a first embodiment of the disclosure. Hereinafter, for convenience of description, one side along a central axis Ax of a sheath 10 is referred to as a distal end side Ar1, while the other side is referred to as a proximal end side Ar2. The ultrasonic treatment system 1 applies treatment energy to a site as a treatment target (hereinafter, referred to as a target site) in a biological tissue, and thereby achieves treatment of the target site. Although the first embodiment is an example in which high frequency energy is adopted in addition to ultrasonic energy as treatment energy, the system may adopt ultrasonic energy alone. As illustrated in FIG. 1, the ultrasonic treatment system 1 includes an ultrasonic treatment tool 2 and a control device 3.

The ultrasonic treatment tool 2 is a surgical treatment tool used for the treatment of a target site through the abdominal wall, for example. The ultrasonic treatment tool 2 includes a handpiece 4 and an ultrasonic transducer 5.

The handpiece 4 includes a holding case 6, a movable handle 7, a first switch 8a, a second switch Bb, a pair of third switches 8c, a rotary knob 9, a sheath 10, a jaw 11, an ultrasonic probe 12 and a cable CA.

The holding case 6 supports the entire ultrasonic treatment tool 2. The holding case 6 includes: a holding case main body 6a having a substantially cylindrical shape and positioned coaxial with the central axis Ax; and a fixed handle 6b extending downward from the holding case main body 6a and gripped by an operator such as a practitioner.

The movable handle 7 receives a closing operation and an opening operation by an operator such as a practitioner. When having received a closing operation by an operator such as a practitioner, the movable handle 7 moves in a direction approaching the fixed handle 6b. In contrast, when having received an opening operation to the movable handle 7, the movable handle 7 moves in a direction away from the fixed handle 6b.

The first and second switches 8a and 8b are provided in a state of being exposed to the outside from the side surface of the distal end side Ar1 of the fixed handle 6b. The first switch 8a receives a setting operation of a first energy output mode by an operator such as a practitioner. The second switch 8b receives a setting operation of a second energy output mode by an operator such as a practitioner. The second energy output mode is an energy output mode different from the first energy output mode.

Here, an example of the first energy output mode is an energy output mode in which coagulation/incision of a target site is performed by applying ultrasonic energy and high frequency energy. In addition, an example of the second energy output mode is an energy output mode in which coagulation of the target site is performed by applying high frequency energy.

The pair of third switches 8c is provided on the front side and the back side (not illustrated) in the drawing in a state of being exposed to the outside of the fixed handle 6b. The pair of third switches 8c receives a setting operation of the third energy output mode by an operator such as a practitioner. Here, examples of the third energy output mode include an energy output mode in which coagulation/incision of a target site is performed by applying ultrasonic energy.

The rotary knob 9 has a substantially cylindrical shape coaxial with the central axis Ax, and is provided on the distal end side Ar1 of the holding case main body 6a. The rotary knob 9 receives a rotation operation by an operator such as a practitioner. By the rotation operation, the rotary knob 9 rotates about the central axis Ax with respect to the holding case main body 6a. The rotation of the rotary knob 9 allows the jaw 11 and the ultrasonic probe 12 to rotate about the central axis Ax.

The sheath 10 has a substantially cylindrical shape as a whole. The sheath 10 has its end on the proximal end side Ar2 attached to the holding case main body 6a.

The jaw 11 is rotatably attached to the end of the sheath 10 on the distal end side Ar1, and grips the target site in cooperation with the end of the ultrasonic probe 12 on the distal end side Ar1. Note that the holding case main body 6a and the sheath 10 described above incorporates an opening/closing mechanism (not illustrated) for opening/closing the jaw 11 with respect to the end on the distal end side Ar1 of the ultrasonic probe 12 in conjunction with the opening/closing operation to the movable handle 7 by the practitioner.

The ultrasonic probe 12 is formed of an electrically conductive material and has an elongated shape extending linearly along the central axis Ax. In addition, the ultrasonic probe 12, in a state where its end on the distal end side Ar1 protrudes to the outside, is inserted into the sheath 10. At this time, an end of the ultrasonic probe 12 on the proximal end side Ar2 is mechanically connected to an ultrasonic transducer 5 described below. That is, the ultrasonic transducer 5 rotates about the central axis Ax together with the ultrasonic probe 12 in conjunction with a rotation operation on the rotary knob 9 by an operator such as a practitioner. The ultrasonic probe 12 then transmits the ultrasonic oscillation generated by the ultrasonic transducer 5 from the end on the proximal end side Ar2 to the end on the distal end side Ar1. In the present embodiment, the ultrasonic oscillation is a vertical oscillation that oscillates in a direction along the central axis Ax.

The cable CA is detachably connected to an electric cable C0 extending from the control device 3. That is, the cable CA is electrically connected to the control device 3 via the electric cable C0.

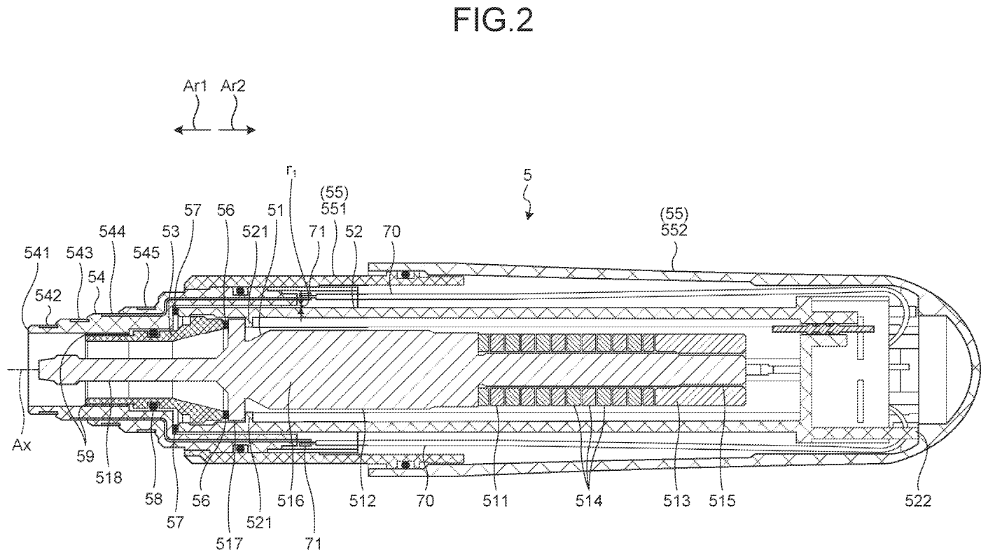

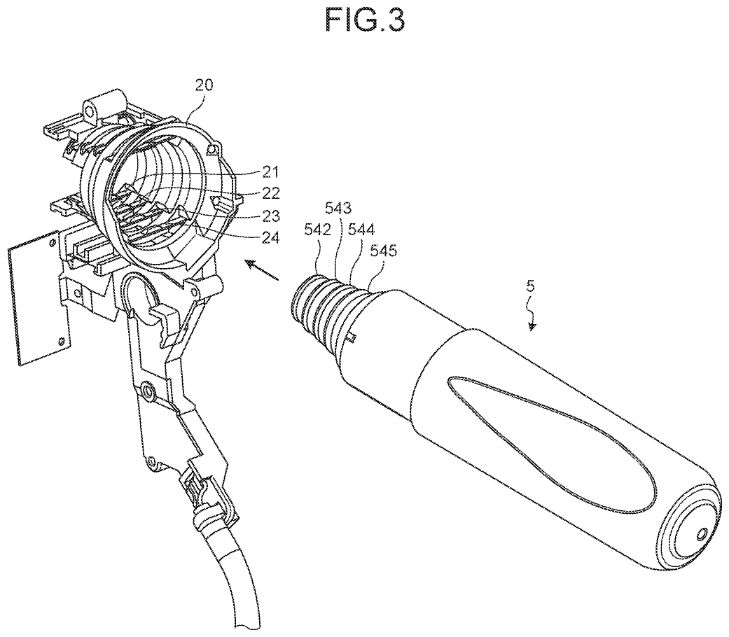

FIG. 2 is a cross-sectional view of the ultrasonic transducer 5 used in the ultrasonic treatment system 1 of FIG. 1. FIG. 3 is a diagram illustrating connection between the ultrasonic transducer 5 and the contact unit.

The ultrasonic transducer 5 includes an ultrasonic oscillator 51, storage 52, a holder 53, a contact receiver 54, and a casing 55.

The ultrasonic oscillator 51 generates ultrasonic oscillation under the control of the control device 3. In the first embodiment, the ultrasonic oscillator 51 is constituted with a Bolted Langevin Transducer (BLT). The ultrasonic oscillator 51 includes an oscillator main body 511, a front mass 512, and a back mass 513.

The oscillator main body 511 includes a plurality of piezoelectric elements 514. Each of the plurality of piezoelectric elements 514 has a disc shape having an opening (not illustrated) at the center, and is stacked along the central axis Ax. In the plurality of piezoelectric elements 514, a potential difference is generated in a stacking direction along the central axis Ax in accordance with a control signal supplied to an electrode plate (not illustrated), causing the piezoelectric elements 514 to alternately repeat expansion and contraction in the stacking direction. This allows the ultrasonic oscillator 51 generates ultrasonic oscillation of vertical oscillation with the stacking direction as the oscillation direction.

The front mass 512 is formed of an electrically conductive material and has an elongated shape extending linearly along the central axis Ax. The front mass 512 includes an element attachment section 515, a cross-sectional area change section 516, a flange 517, and a probe attachment section 518.

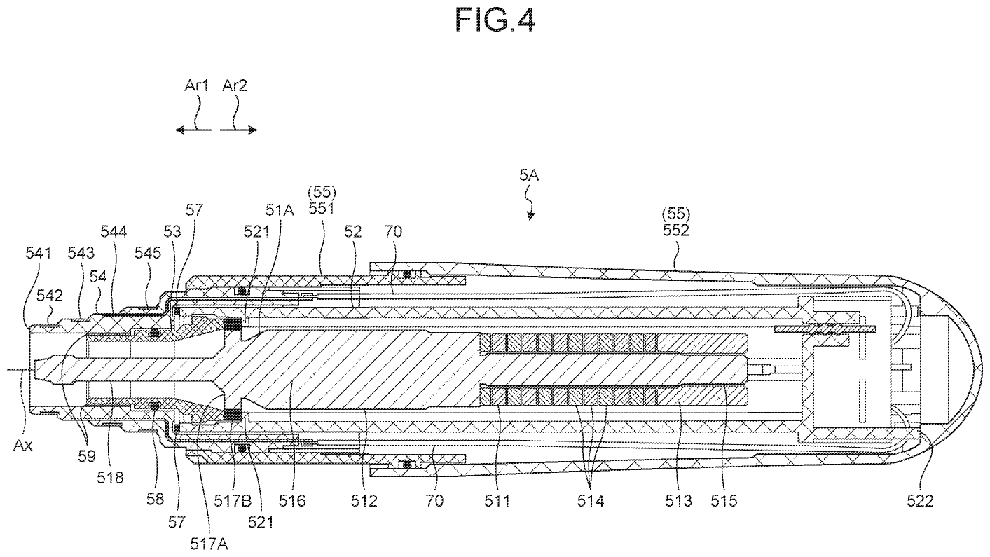

The element attachment section 515 is a bolt extending linearly along the central axis Ax, and is inserted into each opening of a plurality of electrode plates (not illustrated) and each opening (not illustrated) of the plurality of piezoelectric elements 514. As illustrated in FIG. 4, a back mass 513, which is a nut formed of an electrically conductive material, is attached to an end of the element attachment section 515 on the proximal end side Ar2.

The cross-sectional area change section 516 is a section that is provided at an end on the distal end side Ar1 of the element attachment section 515 and that amplifies the amplitude of the ultrasonic oscillation. In addition, the cross-sectional area change section 516 has a truncated cone shape in which the diameter size of the end on the proximal end side Art is set to be larger than that of the element attachment section 515 with the cross-sectional area decreasing as the end on the distal end side Ar1 goes toward the distal end side Ar1. That is, the plurality of piezoelectric elements 514 is grasped between the cross-sectional area change section 516 and the back mass 513 in a state where the element attachment section 515 penetrates along the central axis Ax, allowing the piezoelectric elements 514 to be integrally fastened in a state of having a substantially cylindrical shape.

The flange 517 is provided at an end of the cross-sectional area change section 516 on the distal end side Ar1, and has a diameter size set larger than that of the cross-sectional area change section 516. The flange 517 is sandwiched and fixed between a protrusion 521 of the storage 52 described below and the holder 53. There is provided a seal ring 56 between the flange 517 and the holder 53. With the flange 517 fixed by the storage 52 and the holder 53, the position of the ultrasonic oscillator 51 in the axial direction is fixed.

The probe attachment section 518 is provided at an end of the flange 517 on the distal end side Ar1 and linearly extends along the central axis Ax.

The storage 52, formed of a resin material which is an insulator, has a cylindrical shape and stores the ultrasonic oscillator 51.

The casing 55, formed of a resin material which is an insulator, has a cylindrical shape, externally mounted on an outer side of the storage 52. The casing 55 includes: a first casing 551 as a tubular section located on the distal end side Ar1; and a second casing 552 as a tubular bottomed section located on the proximal end side Ar2.

An end 522 on the proximal end side Ar2 of the storage 52 is in contact with an inner wall of the second casing 552 of the casing 55.

The holder 53, formed of a resin material which is an insulator, has a tubular shape. The proximal end side Ar2 of the holder 53 is inserted into the distal end side Ar1 of the storage 52. The distal end side Ar1 of the holder 53 is inserted into the proximal end side Ar2 of the contact receiver 54 described below.

The contact receiver 54, formed of a resin material which is an insulator and having a cylindrical shape, includes a main body 541 having an outer diameter increasing stepwise from the distal end side Ar1 toward the proximal end side Ar2. In each step section of the main body 541, there are provided a first contact member 542, a second contact member 543, a third contact member 544, and a fourth contact member 545. Each of the first contact member 542, the second contact member 543, the third contact member 544, and the fourth contact member 545 is formed or an electrically conductive material, and is provided over the entire circumference of each step section in the circumferential direction. The proximal end side Ar2 of the contact receiver 54 is inserted into the casing 55. The distal end side Ar1 of the storage 52 is inserted into the proximal end side Ar2 of the contact receiver 54. In addition, a rubber material 57 is disposed between a stepped section 546 on the inner circumference of the contact receiver 54 and the distal end side Ar1 of the storage 52 to be in contact. There is further provided a cable 70 transmitting power and a control signal to the ultrasonic oscillator 51, connected to the proximal end side Ar2 of the contact receiver 54.

A contact unit 20 illustrated in FIG. 3 is provided inside the holding case main body 6a. When the ultrasonic transducer 5 is connected to the holding case main body 6a, the contact receiver 54 of the ultrasonic transducer 5 is inserted into the contact unit 20.

The ultrasonic transducer 5 is inserted into the contact unit 20, and when the operation of the first switch 8a or the second switch 8b causes a first contact member 21, a second contact member 22, a third contact member 23, and a fourth contact member 24 of the contact unit 20 to be connected to the first contact member 542, the second contact member 543, the third contact member 544, and the fourth contact member 545 of the contact receiver 54, respectively, leading to output of ultrasonic energy or high frequency energy.

The distal end side Ar1 of the holder 53 is inserted into the proximal end side Ar2 of the contact receiver 54. There is provided an O-ring 58 disposed between the contact receiver 54 and the holder 53.

The ultrasonic oscillation generated by the ultrasonic oscillator 51 is transmitted to the ultrasonic probe 12 for the treatment of the target site. The generated ultrasonic oscillation is partially transmitted to the storage 52 and the holder 53 via the flange 517. When the ultrasonic oscillation transmitted to the storage 52 and the holder 53 reaches the contact receiver 54, the ultrasonic oscillation can cause a contact failure with the contact unit 20, having a possibility of occurrence of rebooting or overcurrent detection in the generator of the ultrasonic treatment tool 2. Conventionally, the arrangement of the seal ring 56, the rubber material 57, and the O-ring 58 has worked to decrease the transmission of ultrasonic oscillation from the flange 517 to the contact receiver 54 via the storage 52 and the holder 53. However, since the holder 53 and the contact receiver 54 have been bonded and fixed by a hard adhesive, the damping effect of ultrasonic oscillation has not been sufficient.

In the first embodiment, the gap between the contact receiver 54 and the holder 53 in contact with each other is filled with an adhesive 59 formed of a material softer than the holder 53, such as a silicone adhesive, for example, so as to bond and fix the contact receiver 54 and the holder 53 to each other. This makes it possible to dramatically dampen ultrasonic oscillation transmitted from the flange 517 via the holder 53.

Furthermore, in the first embodiment, an interval r1 of the gap between the storage 52 and the contact receiver 54 is set to be larger than the conventional one (0.15 mm to 0.5 mm) so as to further dampen the ultrasonic oscillation transmitted from the flange 517 via the storage 52.

Conventionally, the connection area of the cable 70 connected to the proximal end side Ar2 of the contact receiver 54 is sealed with a sealing resin 71. In this, when the sealing resin 71 and the storage 52 come into contact with each other, ultrasonic oscillation has been transmitted from the storage 52 to the contact receiver 54 via the sealing resin 71. The first embodiment has a configuration in which the sealing resin 71 does not come in contact with the storage 52. With this configuration, it is possible to suppress transmission of ultrasonic oscillation from the storage 52 to the contact receiver 54.

As described above, the first embodiment has a configuration in which the ultrasonic oscillator 51 is in elastic contact with The contact receiver 54, that is, has a floating structure, thereby suppressing transmission of ultrasonic oscillation from the ultrasonic oscillator 51 to the contact receiver 54.

However, when the connection between individual members is made with completely elastic contact, the positional accuracy of The ultrasonic oscillator 51 would decrease. To avoid this, the end 522 on the proximal end side Ar2 of the storage 52 is brought into contact with the inner wall of the second casing 552 of the casing 55 to perform alignment of the ultrasonic oscillator 51. The contact between the end 522 on the proximal end side Ar2 of the storage 52 and the inner wall of the second casing 552 of the casing 55 is rigid contact. Still, the ultrasonic oscillation from the flange 517 via the proximal end side Ar2 of the storage 52 and the second casing 552 of the casing 55 is dampened by the length of the transmission distance, achieving suppression of the influence of the transmission to the contact receiver 54 at a negligible level.

### Second Embodiment

FIG. 4 is a cross-sectional view of an ultrasonic transducer 5A according to a second embodiment of the disclosure. In the ultrasonic transducer 5A according to the second embodiment, a rubber material 517B is integrally molded on an outer circumference of a flange 517A of an ultrasonic oscillator 51A.

As illustrated in FIG. 4, the flange 517A of the ultrasonic oscillator 51A is formed to have a smaller diameter size than the flange 517 of the first embodiment, with the rubber material 517B integrally molded on the outer circumference of the flange 517A. The rubber material 517B is sandwiched and fixed between the protrusion 521 of the storage 52 and the holder 53. In the second embodiment, the rubber material 517B functions as an oscillation damping material that dampens the ultrasonic oscillation transmitted from the ultrasonic oscillator 51A to the storage 52 and the holder 53. Although the second embodiment uses a configuration in which the rubber material 517B is integrally molded with the entire outer circumference of the flange 517A, the rubber material 517B may be integrally molded with a part of the outer circumference of the flange 517A as long as the rubber material 517B can be fixed.

In the second embodiment, the adhesive 59 formed of a softer material than the holder 53 is also used as an oscillation damping material. Still, since a thickness r2 of the rubber material 517B is formed to be the same as the thickness of the flange 517A, with a higher damping effect of ultrasonic oscillation than the seal ring 56 of the first embodiment, it is possible to have a sufficient oscillation damping effect even when the contact receiver 54 and the holder 53 are bonded with a hard adhesive.

### Modification of Second Embodiment

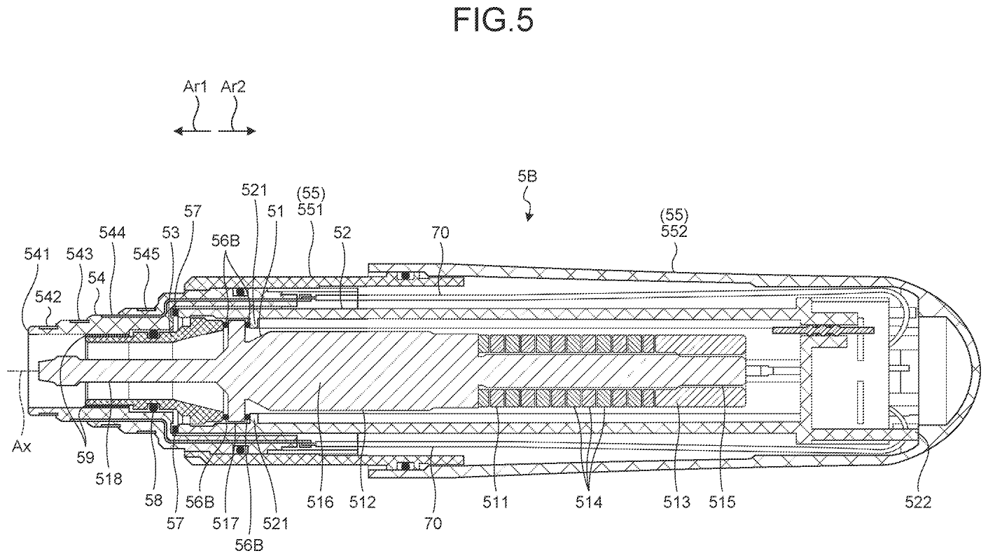

FIG. 5 is a cross-sectional view of an ultrasonic transducer 5B according to a modification of the second embodiment of the disclosure. In the ultrasonic transducer 5B according to the modification of the second embodiment, a ball 56B is used instead of the seal ring 56.

The ball 56B, two pairs of balls 56B are arranged to face each other about the central axis Ax on the distal end side Ar1 and the proximal end side Ar2 of the flange 517 (four balls are arranged on each of the distal end side Ar1 and the proximal end side Ar2). The ball 56B may be an elastic body or a hard body as long as it has a damping function. In the ball 56B, a pair of balls 56B may be arranged to face each other about the central axis Ax on the distal end side An and the proximal end side Ar2 of the flange 517.

### Third Embodiment

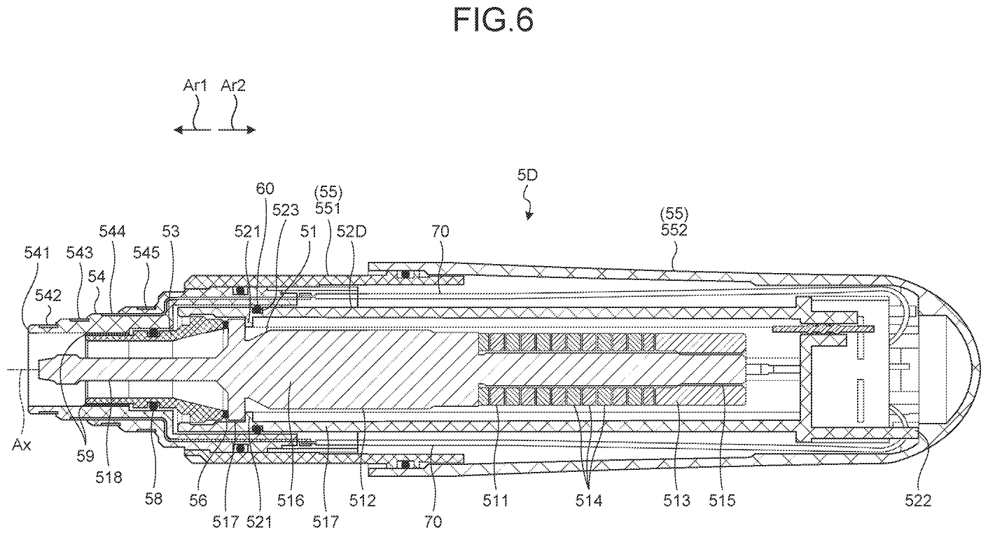

FIG. 6 is a cross-sectional view of an ultrasonic transducer 5D according to a third embodiment of the disclosure. The ultrasonic transducer 5D according to the third embodiment includes an O-ring 60 instead of the rubber material 57 disposed between the contact receiver 54 and the storage 52.

The O-ring 60, disposed so as to be accommodated in a groove 523 provided on the outer circumference of storage 52D, dampens the ultrasonic oscillation transmitted from the flange 517 to the storage 52D and propagating in the radial direction, and suppresses the transmission of the ultrasonic oscillation to the contact receiver 54.

In the third embodiment, the interval r1 of the gap between the storage 52 and the contact receiver 54 is set to be larger than that in the conventional technology (0.15 mm to 0.5 mm), making it possible, with the O-ring 60, to enhance the accuracy of the centering of the ultrasonic oscillator 51.

### Modification of Third Embodiment

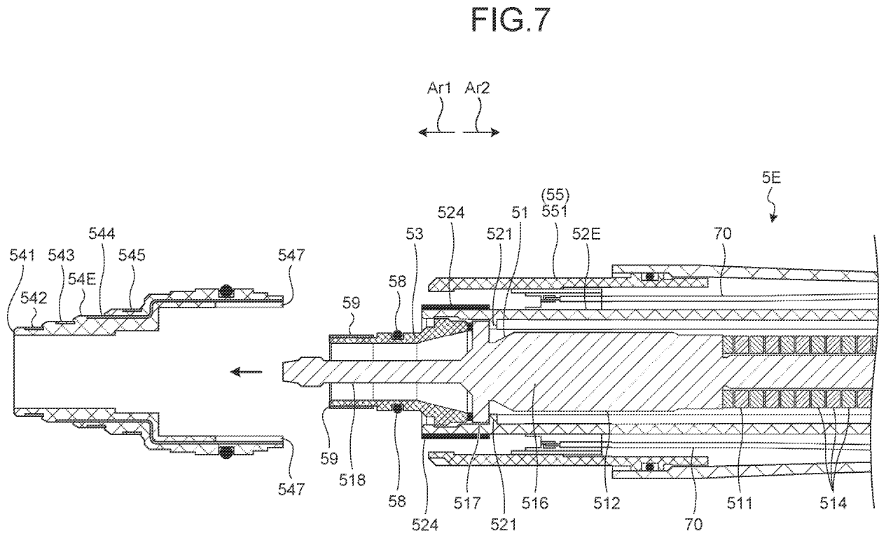

FIG. 7 is a cross-sectional view of an ultrasonic transducer 5E according to a modification of the third embodiment of the disclosure. The ultrasonic transducer 5E according to the modification of the third embodiment includes a rubber material 524 integrally molded on an outer circumference of the distal end side Ar1 of storage 52E, and is provided with a groove 547 on an inner circumference of the proximal end side Ar2 of a contact receiver 54E. The rubber material 524 is fitted to the groove 547 to align the contact receiver 54E and the storage 52E with each other.

In the third embodiment, the rubber material 524 is provided so as to face a part of the outer circumference of the storage 52E (in FIG. 7, disposed to face the vertical direction of storage 52E). However, arrangement is not limited thereto, and the rubber material 524 may be provided on the upper, lower, left, and right, or may be provided on the entire circumference. Moreover, although the groove 547 is provided so as to face a part of the inner circumference of the contact receiver 54E (in FIG. 7, facing the vertical direction of the storage 52E), arrangement is not limited thereto, and the groove 547 may be provided on the upper, lower, left, and right, or may be provided on the entire circumference.

The rubber material 524 can dampen the ultrasonic oscillation transmitted from the flange 517 to the storage 52E and propagating in the radial direction, and can effectively suppress the transmission of the ultrasonic oscillation to the contact receiver 54E.

Furthermore, the third embodiment has a configuration in which the rubber material 524 is fitted in the groove 547 for positioning, making it possible to easily perform positioning not only in the radial direction but also in the axial direction of the ultrasonic oscillator 51.

### Fourth Embodiment

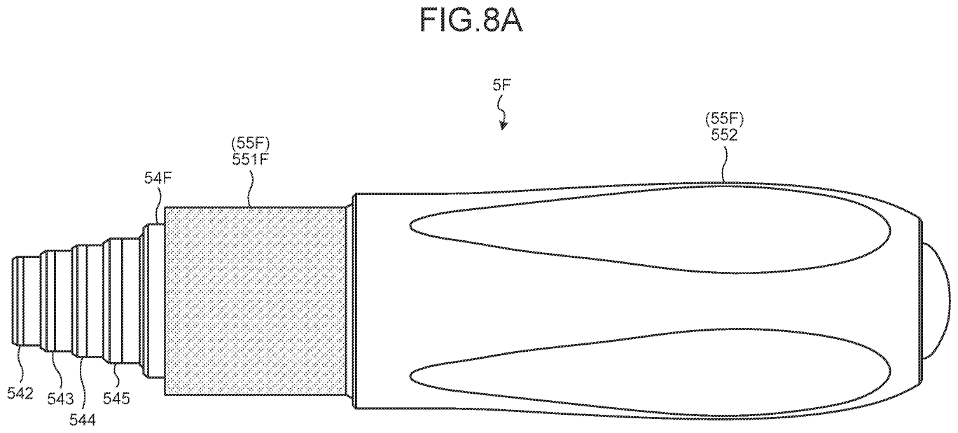

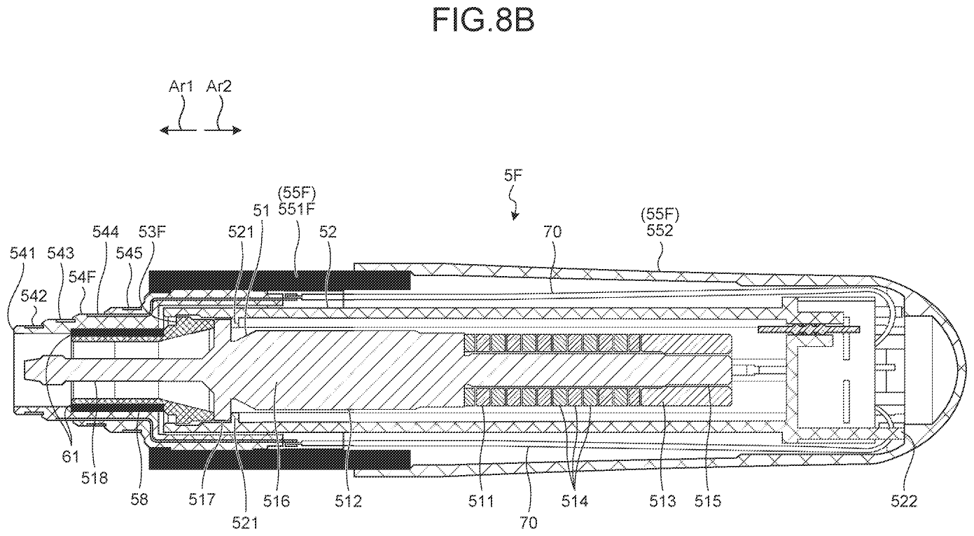

FIG. 8A is a side view of an ultrasonic transducer 5F according to a fourth embodiment of the disclosure. FIG. 8B is a cross-sectional view of the ultrasonic transducer 5F according to the fourth embodiment of the disclosure. In the ultrasonic transducer 5F accord rig to the fourth embodiment, a distal end-side main body 551F of a casing 55E is formed of a rubber material.

In the fourth embodiment, the distal end-side main body 551F of the casing 55F is formed of a rubber material, and a rubber material 61 is further disposed between a holder 53F and a contact receiver 54F. The rubber distal end-side main body 551E and the rubber material 61 can dramatically dampen ultrasonic oscillation transmitted from the flange 517 to the contact receiver 54F.

### Fifth Embodiment

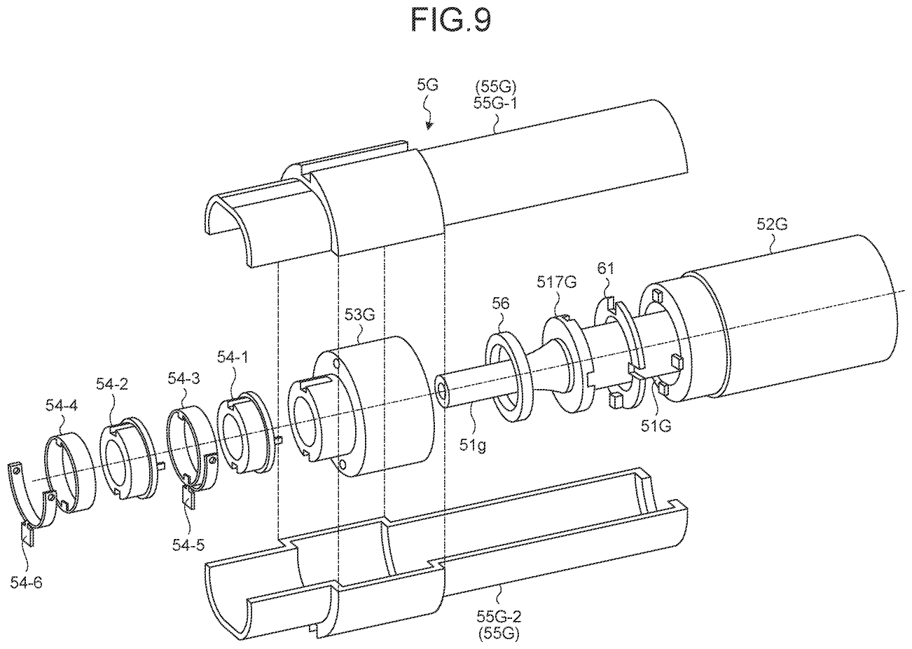

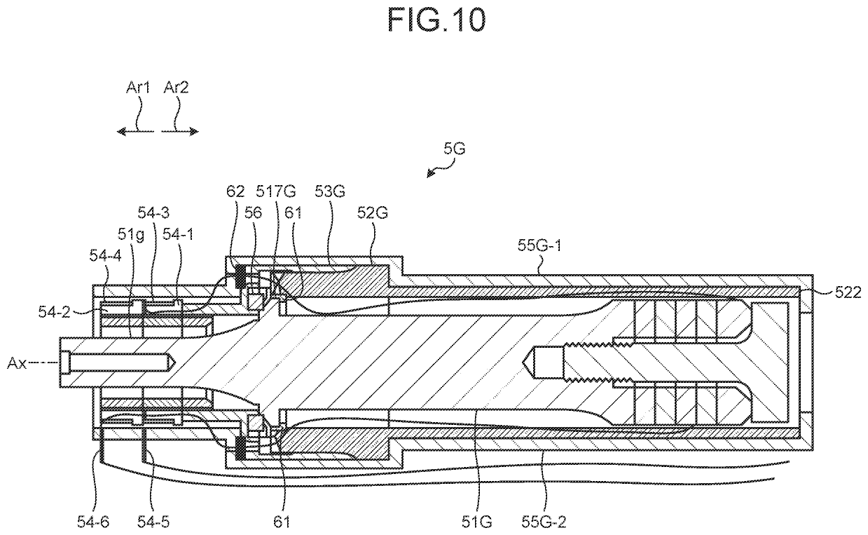

FIG. 9 is an exploded perspective view of an ultrasonic transducer 5G according to a fifth embodiment of the disclosure. FIG. 10 is a cross-sectional view of the ultrasonic transducer 55 according to the fifth embodiment of the disclosure. The ultrasonic transducer 55 includes an ultrasonic oscillator 51G, storage 52G, a holder 53G, and a casing 55G, and also includes a first drum engagement 54-3, a second drum engagement 54-4, a first housing engagement 54-5, and a second housing engagement 54-6, which function as contacts.

The ultrasonic oscillator 51G is stored in the storage 52G. The storage 52G is externally covered with a casing 55G including a first case 55G-1 and a second case 55G-2.

The end 522 of the proximal end side Ar2 of the storage 52G is in contact with an inner wall of the proximal end side Ar2 of the casing 55G.

The distal end side Ar1 of the storage 52G is inserted into the proximal end side Ar2 of the holder 53G. The inner circumferential surface of the holder 53G and the distal end side Ar1 of the storage 52G sandwich and hold a flange 517G of the ultrasonic oscillator 51G. There is an O-ring disposed between the holder 53G and the flange 517G, with a keyed ring 61G disposed between the flange 517G and the storage 52G.

A first drum housing 54-1 and a second drum housing 54-2 are fixed to the distal end side Ar1 of the holder 53G by a support column and a support column hole (not illustrated). In addition, the first drum engagement 54-3 and the second drum engagement 54-4 are respectively fitted to the outer circumference of the first drum housing 54-1 and the second drum housing 54-2.

The first housing engagement 54-5, equipped with a downwardly extending tab connected to a half ring, forms an elastically biased stripping engagement so as to bring the half ring into contact with the first drum engagement 54-3.

The second housing engagement 54-6, equipped with a downwardly extending tab connected to a half ring, forms an elastically biased stripping engagement so as to bring the half ring into contact with the second drum engagement 54-4.

The first drum engagement 54-3, the second drum engagement 54-4, the first housing engagement 54-5, and the second housing engagement 54-6 are each formed of an electrically conductive material and function as contacts.

The first drum housing 54-1, the second drum housing 54-2, the first drum engagement 54-3, the second drum engagement 54-4, the first housing engagement 54-5, and the second housing engagement 54-6 correspond to the contact receivers.

In the fourth embodiment, a rubber material 62 is disposed on the distal end side An of the holder 53G in contact with the casing 55G. The rubber material 62 makes it possible to dampen the ultrasonic oscillation transmitted from the flange 517G via the holder 53G.

### Sixth embodiment

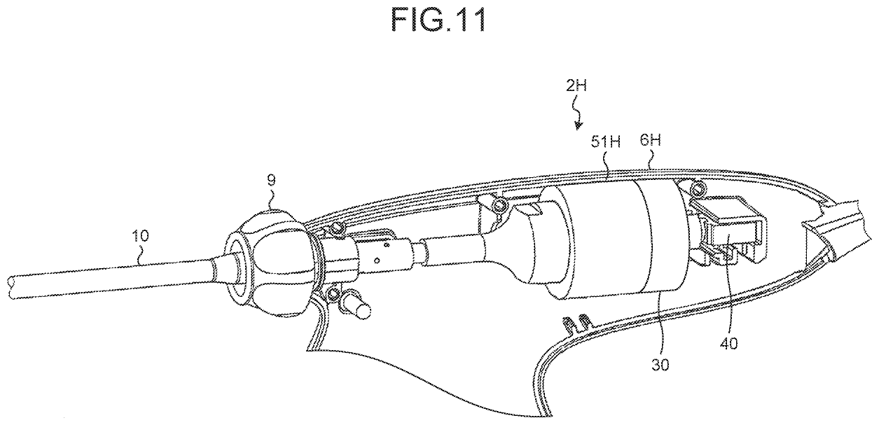

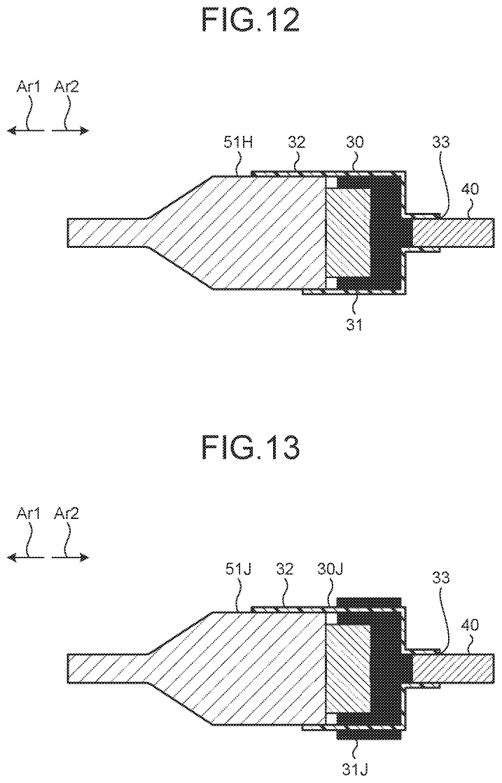

FIG. 11 is a partial cross-sectional view of an ultrasonic treatment tool 2H according to a sixth embodiment of the disclosure. FIG. 12 is a cross-sectional view of an ultrasonic oscillator 51H and a cover 30 used in the ultrasonic treatment tool 2H of FIG. 11. The ultrasonic treatment tool 2H according to the sixth embodiment is a disposable ultrasonic treatment tool in which the ultrasonic oscillator 51 is directly mounted in a holding case OH of the ultrasonic treatment tool 2H.

As illustrated in FIG. 12, the cover 30 includes: a main body 31 that is formed of a rubber material and covers the proximal end side Ar2 of the ultrasonic oscillator 51H; and a flexible substrate 32 that covers the outer circumference of the main body 31 and transmits power and a control signal to the ultrasonic oscillator 51H.

Flexible substrate 32 has an opening 33 on the proximal end side Ar2, with an earphone jack 40 being inserted into the opening 33. By connecting the earphone jack 40 to a connector 41 in the holding case 6H, power and a control signal are transmitted to the ultrasonic oscillator 51H via the flexible substrate 32. In the sixth embodiment, the earphone jack 40 corresponds to the contact receiver.

In the sixth embodiment, the ultrasonic oscillation generated by the ultrasonic oscillator 51H is dampened by the main body 31 of the cover 30, making it possible to suppress transmission of the ultrasonic oscillation to the connector 41.

### First Modification of sixth Embodiment

FIG. 13 is a cross-sectional view of an ultrasonic oscillator 513 and a cover 30J according to a first modification of the sixth embodiment. In the first modification of the sixth embodiment, the size of a main body 31J in the radial direction is formed to be larger than the size of the flexible substrate 32 in the radial direction.

The cover 30J can be manufactured by inserting the flexible substrate 32 into the main body 31J or insert-molding a rubber material to be a material of the main body 31J around the flexible substrate 32.

In the first modification of the sixth embodiment, the ultrasonic oscillation generated by the ultrasonic oscillator 51J is dampened by the main body 31J of the cover 30J, making it possible to suppress transmission of the ultrasonic oscillation to the connector 41.

### Second Modification of Sixth embodiment

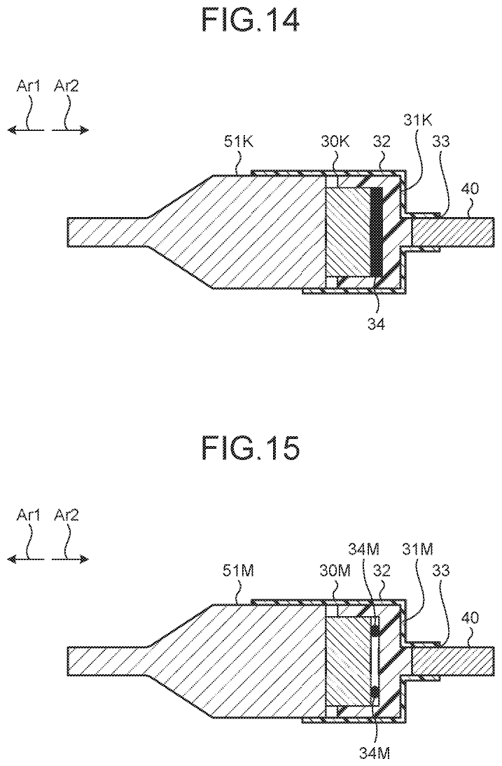

FIG. 14 is a cross-sectional view of an ultrasonic oscillator 51K and a cover 30J according to a second modification of the sixth embodiment. In the second modification of the sixth embodiment, a main body 31K is formed of a hard resin, and a rubber material 34, which is an oscillation damping material, is disposed between the ultrasonic oscillator 51K and the main body 31K.

In the second modification of the sixth embodiment, the ultrasonic oscillation generated by the ultrasonic oscillator 51K is dampened by the rubber material 34 of a cover 30K, making it possible to suppress transmission of the ultrasonic oscillation to the connector 41.

In addition, since the main body 31K is formed of a hard resin, excellent durability is achieved.

### Third Modification of Sixth Embodiment

FIG. 15 is a cross-sectional view of an ultrasonic oscillator 51M and a cover 30K according to a third modification of the sixth embodiment. In the third modification of the sixth embodiment, a main body 31M is formed of a hard resin, and a ball 34M being an oscillation damping material is disposed between the ultrasonic oscillator 51M and the main body 31K.

In the third modification of the sixth embodiment, the ultrasonic oscillation generated by the ultrasonic oscillator 51M is dampened by the ball 34M of the cover 30M, making it possible to suppress transmission of the ultrasonic oscillation to the connector 41.

In addition, since the main body 31M is formed of a hard resin, excellent durability is achieved.

### Seventh Embodiment

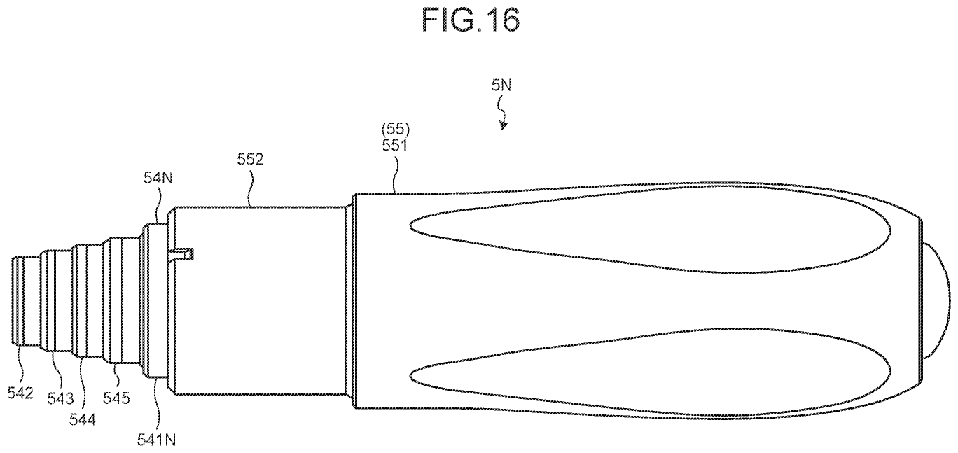

FIG. 16 is a side view of an ultrasonic transducer 5N according to a seventh embodiment of the disclosure. In the ultrasonic transducer 5N according to the seventh embodiment, a main body 541N of a contact receiver 54N is formed of a rubber material.

In the seventh embodiment, the ultrasonic oscillation generated by the ultrasonic oscillator is dampened by the main body 541N of the contact receiver 54N, making it possible to prevent a contact failure between the contact receiver 54N and the contact unit.

The ultrasonic treatment tool and the ultrasonic transducer of the disclosure includes the oscillation damping material that prevents transmission of the ultrasonic oscillation to the contact receiver, making it possible to suppress a contact failure between the contact receiver and the contact unit.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment tool comprising:
an ultrasonic oscillator in which a plurality of piezoelectric elements configured to generate ultrasonic oscillation are arranged along a longitudinal axis direction of the ultrasonic oscillator, the ultrasonic oscillator including a flange at a node position of ultrasonic oscillation in a resonance state, the ultrasonic oscillator being configured to transmit the ultrasonic oscillation from a proximal end side to a distal end side of the ultrasonic oscillator;
a contact receiver configured to transmit power and a control signal to the ultrasonic oscillator; and
a floating structure configured to suppress transmission of ultrasonic oscillation generated by the ultrasonic oscillator to the contact receiver or dampen the ultrasonic oscillation,
the floating structure including:
an oscillation damping material configured to dampen the ultrasonic oscillation transmitted from the flange to the contact receiver;
a storage configured to accommodate the ultrasonic oscillator; and
a holder that is inserted into the contact receiver and the storage, the holder being configured to hold the flange of the ultrasonic oscillator in cooperation with the storage,
the oscillation damping material being formed of a material that is softer than the holder and that is filled in a gap at a portion where the contact receiver and the holder are is contact with each other.

2. The ultrasonic treatment tool according to claim 1, wherein the oscillation damping material is made of a silicone adhesive.

3. The ultrasonic treatment tool according to claim 1, further comprising
a casing configured to accommodate a proximal end side of the contact receiver and accommodate the storage,
wherein at least a part of a proximal end side of the storage is in contact with a proximal end-side inner wall of the casing.

4. The ultrasonic treatment tool according to claim 1, further comprising: an ultrasonic transducer including the ultrasonic oscillator and the contact receiver; and a handpiece to which the ultrasonic transducer is detachable.

5. The ultrasonic treatment tool according to claim 4, wherein the handpiece is provided with a contact unit to which the contact receiver is attached.

6. The ultrasonic treatment tool according to claim 4, wherein high frequency energy is supplied to the ultrasonic transducer.

7. An ultrasonic treatment tool comprising:
an ultrasonic oscillator in which a plurality of piezoelectric elements configured to generate ultrasonic oscillation are arranged along a longitudinal axis direction of the ultrasonic oscillator, the ultrasonic oscillator being configured to transmit the ultrasonic oscillation from a proximal end side to a distal end side of the ultrasonic oscillator;
a contact receiver configured to transmit power and a control signal to the ultrasonic oscillator; and
a floating structure configured to suppress transmission of ultrasonic oscillation generated by the ultrasonic oscillator to the contact receiver or dampen the ultrasonic oscillation,
the floating structure including a cover that includes an oscillation damping material, the cover being configured to hold the proximal end side of the ultrasonic oscillator,
the cover being configured to hold an earphone jack functioning as the contact receiver on a side facing a side holding the ultrasonic oscillator,
the cover including:
a main body formed of a rubber material and covering the proximal end side of the ultrasonic oscillator; and
a flexible substrate covering an outer circumference of the main body, the flexible substrate being configured to transmit the power and the control signal to the ultrasonic oscillator.

8. An ultrasonic treatment tool comprising:
an ultrasonic oscillator in which a plurality of piezoelectric elements configured to generate ultrasonic oscillation are arranged along a longitudinal axis direction of the ultrasonic oscillator, the ultrasonic oscillator being configured to transmit the ultrasonic oscillation from a proximal end side to a distal end side of the ultrasonic oscillator;
a contact receiver configured to transmit power and a control signal to the ultrasonic oscillator; and
a floating structure configured to suppress transmission of ultrasonic oscillation generated by the ultrasonic oscillator to the contact receiver or dampen the ultrasonic oscillation,
the floating structure including a cover that includes an oscillation damping material, the cover being configured to hold the proximal end side of the ultrasonic oscillator,
the cover being configured to hold an earphone act functioning as the contact receiver on a side facing a side holding the ultrasonic: oscillator,
the cover including:
a main body formed of a resin and covering the proximal end side of the ultrasonic oscillator;
a flexible substrate covering an outer circumference of the main body, the flexible substrate being configured to transmit the power and the control signal to the ultrasonic oscillator; and
the oscillation damping material disposed between the ultrasonic oscillator and the main body.

9. The ultrasonic treatment tool according to claim 7, wherein the contact receiver is formed of a rubber material.

10. The ultrasonic treatment tool according to claim 8, wherein the contact receiver is formed of a rubber material.

11. The ultrasonic treatment tool according to claim 7, wherein the ultrasonic oscillator and the contact receiver are disposed in a holding case.

12. The ultrasonic treatment tool according to claim 8, wherein the ultrasonic oscillator and the contact receiver are disposed in a holding case.
